(19) United States
(12) Patent Application Publication
(10) Pub. No.: US 2023/0407323 A1
(43) Pub. Date: Dec. 21, 2023

(54) DELIVERY OF GENOME EDITING REAGENTS VIA CHIMERIC PLANTS

(71) Applicant: Calyxt, Inc., Roseville, MN (US)

(72) Inventor: Nathaniel Butler, Roseville, MN (US)

(73) Assignee: Calyxt, Inc., Roseville, MN (US)

(21) Appl. No.: 18/248,010

(22) PCT Filed: Oct. 7, 2021
(86) PCT No.: PCT/US2021/053985

§ 371 Date: Apr. 5, 2023

Related U.S. Application Data
(60) Provisional application No. 63/088,790, filed on Oct. 7, 2020.

(51) Int. Cl. C12N 15/82

(52) U.S. Cl. CPC C12N 15/8213; C12N 15/8202

(57) ABSTRACT

Embodiments of the present disclosure are directed to a method that includes generating a chimeric plant by infecting a cutting of a host plant with Rhizobium rhizogenes carrying an expression construct, and screening new growth from the chimeric plant for gene edits resulting from genomic editing by the expression construct.

### BACKGROUND

Methods of genome editing, which are performed without the use of deoxyribonucleic acid (DNA) for mutation initiation, are referred to as “DNA-free” genome editing techniques. DNA-free genome editing in plants typically requires the direct delivery of transgenic ribonucleic acid (RNA) and/or protein from genome editing reagents to plant cells and regeneration of transformed cells as whole plants and edited lines. This approach requires specialized plant transformation and tissue culture protocols to deliver transgenic RNA and/or protein directly to plant tissues and regenerate transformed material as whole plants. In some cases, RNA viruses have been used to deliver genome editing reagents, such as single guide RNAs (sgRNAs) or small RNAs. However, strict viral size limits prevent larger reagents, such as transcription activator like effector nucleases (TALENs) or Crisper associated protein 9 (Cas9) to be incorporated directly into viral particles. Furthermore, “transient” methods of reagent delivery incorporating protoplasts, Agrobacterium tumefaciens, Rhizobium rhizogenes or geminivirus replicons (GVRs) have also been demonstrated but require costly tissue culture protocol development and risk incorporation of DNA into the host plant.

### SUMMARY

This summary is provided to introduce a selection of concepts in a simplified form that are further described below in the Detailed Description.

In one aspect, the disclosure provides a method of delivery of genome editing reagents. The method comprises generating a chimeric plant by infecting a cutting of a host plant with Rhizobium rhizogenes (R. rhizogenes) carrying an expression construct, and screening new growth from the chimeric plant for gene edits resulting from genomic editing by the expression construct. Rhizobium rhizogenes may also be referred to as Agrobacterium rhizogenes (A. rhizogenes), and the terms may be used interchangeably herein.

In some examples, the chimeric plant includes a wild type/transgenic chimeric plant. In some examples, the expression construct includes a transcription activator like effector nuclease (TALEN) mRNA. In some examples, the expression construct includes an mRNA coding sequence, and a promoter. In some examples, the promoter is 35S. In some examples, the promoter is nopaline synthase (Nos).

In some examples, the method includes screening the new growth from the chimeric plant with detectable transgenic mRNA or protein from the expression construct and/or for gene edits resulting from genomic editing and propagating tissue or seed from the tissue with detectable transgenic mRNA or protein or genomic edits.

In another example embodiment, a method of generating chimeric plants expressing genome editing reagents includes preparing a wild-type shoot or seedling cutting from a host plant, inoculating the wild-type shoot or seedling cutting with a Rhizobium rhizogenes solution, and inoculating the wild-type shoot or seedling cutting with a Rhizobium rhizogenes solution, and transferring the wild-type shoot or seeding infected with the R. rhizogenes solution to a medium for growth of transgenic hairy roots.

In some examples, the method includes screening and selecting individual chimeric plants for expression of transcription activator like effector nuclease (TALEN) mRNA and/or protein. In some examples, the method includes detecting accumulation of transgenic RNA and/or protein in shoot tissues using end-point reverse transcriptase PCR (RT-PCR) or western blot. Various examples are directed toward a non-naturally occurring plant generated by the method, and a non-naturally occurring seed, reproductive tissue, or vegetative tissue generated by the method.

In another aspect, the disclosure provides a non-naturally occurring plant, generated by the genomic editing technique. In such embodiments, the genomic editing technique includes preparing a cutting of a host plant and generating a chimeric plant by infecting the cutting of the host plant with an R. rhizogenes solution including a TALEN mRNA coding sequence, and a promoter. The genomic editing technique further includes screening growth from the chimeric plant resulting from genomic editing by the TALEN mRNA and/or protein, and propagating the growth or seed from the chimeric plant with accumulation of the TALEN mRNA and/or protein for isolation of edited events.

In some examples, the TALEN mRNA targets a Cannabis sativa tetrahydrocannabinolic acid synthase (CsTHC) gene. In some examples, the TALEN mRNA targets a Solanum tuberosum phytoene desaturase (StPDS) gene.

### DESCRIPTION OF THE DRAWINGS

The foregoing aspects and many of the attendant advantages of this invention will become more readily understood by reference to the following detailed description, when taken in conjunction with the accompanying drawings, wherein:

FIG. 1 illustrates an example method for delivery of genome editing reagents via chimeric plants, consistent with the present disclosure.

FIG. 2 illustrates an example method for delivery of genome editing reagents via chimeric plants, consistent with the present disclosure.

FIGS. 3A and 3B illustrate example expression constructs for delivery of genome editing reagents, consistent with the present disclosure.

FIGS. 4A and 4B illustrate growth of transgenic hairy roots in potato, consistent with the present disclosure.

FIGS. 5A, and 5B illustrate growth of transgenic hairy roots in hemp, consistent with the present disclosure.

FIG. 6 illustrates chimeric hemp plant with transgenic hairy roots, consistent with the present disclosure.

FIG. 7 illustrates chimeric potato plant with transgenic hairy roots, consistent with the present disclosure.

FIGS. 8A and 8B illustrate gel electrophoresis results obtained from chimeric potato plant tissue, consistent with the present disclosure.

FIG. 9 illustrates development of hemp transgenic hairy roots, consistent with the present disclosure.

FIG. 10 illustrates data obtained from shoot tips of chimeric potato plants, consistent with the present disclosure.

### DETAILED DESCRIPTION

Various genomic editing methods allow for DNA-free editing in plants. These methods include direct delivery of RNA and/or protein, “transient” DNA delivery via protoplasts, Agrobacterium tumefaciens, Rhizobium rhizogenes, or particle bombardment, and viral delivery of RNA reagents (i.e. sgRNAs and small RNAs) or DNA (i.e. geminivirus replicons). All of these methods require some degree of reagent delivery to the target plant genotype and regeneration of edited tissues. Furthermore, these delivery methods are often inefficient and require preparation of RNA, protein and viral particles which may further reduce efficiencies.

Plant transformation and tissue culture techniques present significant limitations to genome editing efforts, requiring extensive time, labor and materials to develop and implement specialized protocols. DNA-free editing techniques discussed herein may save time by eliminating the incorporation of transgenic DNA. Delivery of genome editing reagents via chimeric plants, consistent with the present disclosure, enables direct delivery of DNA-free genome editing reagents to plant tissues and may expand the number of plant genotypes capable of being edited. Moreover, generation of chimeric plants with transgenic hairy roots via R. rhizogenes provides an opportunity to deliver RNA and/or protein from genome editing reagents without direct transformation and regeneration of plant materials and may allow for genome editing in recalcitrant and non-recalcitrant plant genotypes.

The vascular system of plants allows for the transportation of water and minerals (via the xylem) and sugars (via the phloem) to growing parts of the plant (i.e., sinks). Macromolecules such as RNA and protein are also transported through the vascular system (primarily the phloem) for long-distance signaling and control multiple plant functions. Long-distance signaling works by the synthesis of macromolecules in the companion cells of the phloem in leaves and roots (i.e., sources), and the loading macromolecules into sieve elements for transport to distant growing parts of the plant (i.e., sinks) via the plasmodesmata. As disclosed herein, expression of genome editing reagents in phloem companion cells in transgenic hairy roots resulting from R. rhizogenes infection may facilitate long-distance transport of transgenic RNA and/or protein and DNA-free editing in sink tissues for propagation and isolation of individual edited events.

Many benefits are achieved by delivery of genome editing reagents via chimeric plants according to the present disclosure. First, direct transformation and regeneration of target genotypes is not required for developing editing events in target genotype backgrounds, which reduces the cost of developing and implementing genotype-specific transformation and regeneration protocols. Second, preparation of DNA-free reagents, such as RNA and protein is not required, which further reduces the production cost and technical development of such dedicated protocols. Third, once chimeric lines with transgenic roots expressing genome editing reagents have been developed, tissue culture is not required for delivery of the genome editing reagents, and production may be conducted in non-tissue culture environments. Advantages of the present disclosure are not limited to those enumerated above, and additional benefits may be realized.

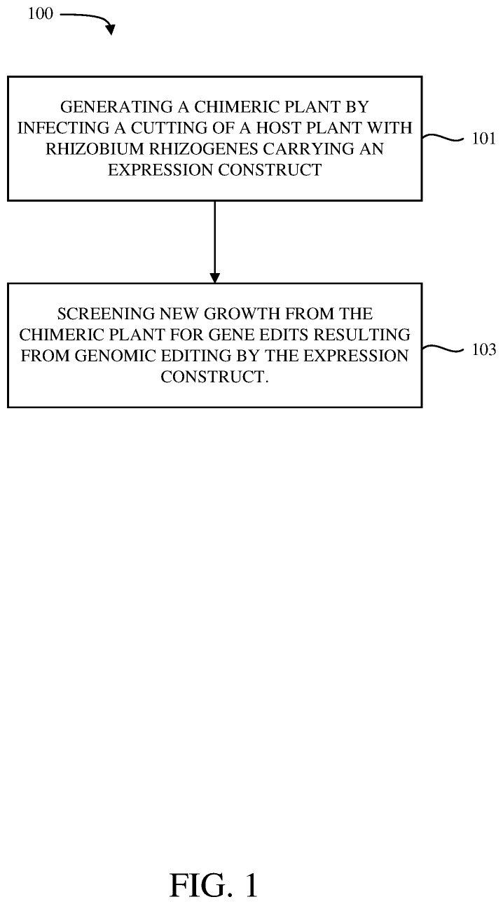

FIG. 1 illustrates an example method 100 for delivery of genome editing reagents via chimeric plants, consistent with the present disclosure. R. rhizogenes is a Rhizobium species used to transform plant cells and that results in high virulence and rapid development of transgenic materials in the form of hairy roots. Hairy roots resulting from R. rhizogenes infection of plant stem tissue carry the transfer DNA (T-DNA) from the so-called root-inducing (Ri) plasmid and form vascular connections with their plant hosts. These vascular connections allow the hairy roots to function similarly to wild-type roots, and may grow aggressively, out-competing wild-type roots. In addition to the T-DNA from the Ri plasmid, additional T-DNAs, such as those from binary vectors carrying genome editing reagents, may be co-delivered to plant stem tissues and may be expressed in hairy roots as transgenic hairy roots. TALEN is one example of a binary vector carrying genome editing reagents that may be co-delivered to the plant tissue and is discussed further therein.

Transgenic hairy roots that form on a wild-type plant host create a transgenic/wild-type chimeric plant capable of expressing genome editing reagents in the form of RNA and/or protein in transgenic hairy root tissues. If transgene expression is directed to phloem tissues (i.e., companion cells) of transgenic hairy roots, transgenic RNA and/or protein may be loaded into the phloem sieve elements and transported long-distances to growing wild-type tissues. Accumulation of transgenic RNA and/or protein in growing tissues may result in editing in the absence of transgenic DNA, and individual events may be isolated by vegetative propagation and/or production of seed.

Method 100 at step 101 includes generating a chimeric plant by infecting a cutting of a host plant with R. rhizogenes carrying an expression construct. Chimeric plants expressing TALENs or other genome editing reagents may first be generated in a genotype or species susceptible to R. rhizogenes. The method used for R. rhizogenes infection may vary depending on the species of the host plant, but typically includes the preparation of a fresh wild-type shoot (cut at the stem) or seedling (cut at the hypocotyl) cuttings, and inoculation of the cut end with a R. rhizogenes solution. Accordingly, the chimeric plant may be a wild-type/transgenic chimeric plant. Co-cultivation of the chimeric plant for approximately two-days on non-selective media may facilitate delivery of both Ri plasmid and binary vector T-DNAs to the wild-type tissue.

As used herein, an expression construct refers to or includes a nucleic acid sequence including one or more binary vectors carrying genome editing reagents (such as TALEN mRNA), and a promoter. The term ‘promoter’ refers to or includes a sequence of DNA that turns a gene on or off. In some examples, the promoter may be a constitutive promoter that is active in vivo, an inducible promoter that may be turned on and/or off, or phloem-specific promoter that has activity in phloem tissues. In some examples, the expression construct may include one or more zip-code elements. As used herein, a zip-code element refers to or includes cis-acting signals, or ‘zip-codes’, that permit mRNA sequences to transport through the phloem (e.g., a phloem-mobile RNA). A zip-code element may include an untranslated region (UTR) from the phloem-mobile RNA, or the full-length sequence from a phloem-mobile RNA. Non-limiting examples of a zip-code element include gibberellic acid insensitive (GAI) mRNA such as Arabidopsis GAI, and knotted1-like homeobox (KNOX) mRNA, Tomato KNOTTED1 (LeT6), Potato BEL5, Arabidopsis tRNAmet (At5g57885), Arabidopsis tRNAgly (At5g57885), Arabidopsis CENTRORADIALIS (ATC), Potato KNOTTED1 (StPOTH1), Pumpkin NACP (NAM, ATAF1/3 and CUC2), and Pumpkin GAIP, among others. Various embodiments in accordance with the present disclosure can include at least some of substantially the same features and attributes, including zip-code elements and gene sequences, as discussed in the following references, each of which are hereby incorporated by reference in their entireties for their general teachings related to zip-code elements and the specific teachings related to the sequence(s) of the particular genes: Huang, NC.; Yu, TS. The sequences of Arabidopsis GA-INSENSITIVE RNA constitute the motifs that are necessary and sufficient for RNA long-distance trafficking. Plant J. 2009, 59, 921-929; Kim, M.; Canio, W.; Kessler, S.; Sinha, N. Developmental changes due to long distance movement of a homeobox fusion transcript in tomato. Science 2001, 293, 287-289; Banerjee, A. K.; Chatterjee, M.; Yu, Y.; Suh, S. G.; Miller, W. A.; Hannapel, D. J. Dynamics of a mobile RNA of potato involved in a long-distance signaling pathway. Plant Cell 2006, 18, 3443-3457; Li, C.; Gu, M.; Shi, N.; Zhang, H.; Yang, X.; Osman, T.; Liu, Y.; Wang, H.; Vatish, M.; Jackson, S.; et al. Mobile FT mRNA contributes to the systemic florigen signalling in floral induction. Sci. Rep. 2011, 1, 73; Zhang, W.; Thieme C. J.; Kollwig G.; Apelt, F.; Yang, Lei.; Winter, N.; Andresen, N.; Walther, D.; Kragler, F. tRNA-Related Sequences Trigger Systemic mRNA Transport in Plants. Plant Cell 2016, 28, 1237-1249; Huang, N.C.; Jane, W. N.; Chen, J.; Yu, T. S. Arabidopsis CENTRORADIALIS homologue acts systemically to inhibit floral initiation in Arabidopsis. Plant J. 2012, 72, 175-184; Mahajan, A.; Bhogle, S.; Kang, I. H.; Hannapel, D. J.; Banerjee, A. K. The mRNA of a Knotted1-like transcription factor of potato is phloem mobile. Plant Mol. Biol. 2012, 79, 595-608; Ruiz-Medrano, R.; Xoconostle-Cazares, B.; Lucas, W. J. Phloem long-distance transport of CmNACP mRNA: Implications for supracellular regulation in plants. Development 1999, 126, 4405-4419; Haywood, V.; Yu, T. S.; Huang, N.C.; Lucas, W. J. Phloem long-distance trafficking of GIBBERELLIC ACID-INSENSITIVE RNA regulates leaf development. Plant J. 2005, 42, 49-68.

Also as used herein, a phloem-specific promoter refers to or includes a promoter that targets phloem-specific gene expression. These promoter elements may be associated with genes that are expressed specifically in phloem cells or from organisms that are phloem limited. Non-limiting examples of phloem-specific promoters include sucrose transport protein 1 (SUT1), figwort mosaic virus (FMV), sucrose transport protein 2 (SUC2), Arabidopsis SUC2, Tomato SUT1, Potato PTB1, and Agrobacterium rolC. Also as used herein, a constitutive promoter refers to or includes a promoter that targets phloem-specific gene expression. These promoter elements may be associated with genes that are expressed across plant cell types and may come from non-plant sources. Non-limiting examples of constitutive promoters include 35S promoter, 2× 35S promoter, nopaline synthase (Nos) promoter, VaUbi3, among others. Also as used herein, an inducible promoter refers to or includes a promoter that targets phloem-specific gene expression. These promoter elements may be associated with gene expression induced by exposure to an exogenous factor (e.g., β-estradiol) and may come from non-plant sources. Non-limiting examples of inducible promoters include, P16ΔS:sXVE promoter, and SUPERR:sXVE promoter, among others. Various embodiments in accordance with the present disclosure can include at least some of substantially the same features and attributes, including promoters and gene sequences, as discussed in the following references, each of which are hereby incorporated by reference in their entireties for their general teachings related to plant genetics and the specific teachings related to the sequence(s) of the particular promoters: Srivastava, A. C.; Ganesan, S.; Ismail, I. O.; Ayre, B. G. Functional Characterization of the Arabidopsis AtSUC2 Sucrose/H+ Symporter by Tissue-Specific Complementation Reveals an Essential Role in Phloem Loading but Not in Long-Distance Transport. Plant Physiol. 2008, 148, 200-211; Kuhn, C.; Hajirezaei, M. R.; Fernie, A. R., Roessner-Tunali, U.; Czechowski.; Hirner, B.; Frommer, W. B. The Sucrose Transporter StSUT1 Localizes to Sieve Elements in Potato Tuber Phloem and Influences Tuber Physiology and Development. Plant Physiol. 2003, 131, 102-113; Butler, N. M.; Hannapel, D. J. Promoter activity of polypyrimidine tract-binding protein genes of potato responds to environmental cues. Planta 2012, 236, 1747-1755; Schmiilling, T.; Schell, J.; Spena, A. Promoters of the rolA, B, and C Genes of Agrobacterium rhizogenes are Differentially Regulated in Transgenic Plants. Plant Cell 1989, 1, 665-670; Schluckinga, K.; Edel, K. H.; Drerup, M. M.; Köster, P.; Eckert, C.; Steinhorst, L., Waadt, R.; Batisti\grave{c}, O.; Kudla, J. A New β-Estradiol-Inducible Vector Set that Facilitates Easy Construction and Efficient Expression of Transgenes Reveals CBL3-Dependent Cytoplasm to Tonoplast Translocation of CIPK5. Mole. Plant 2013, 6, 1814-1829.

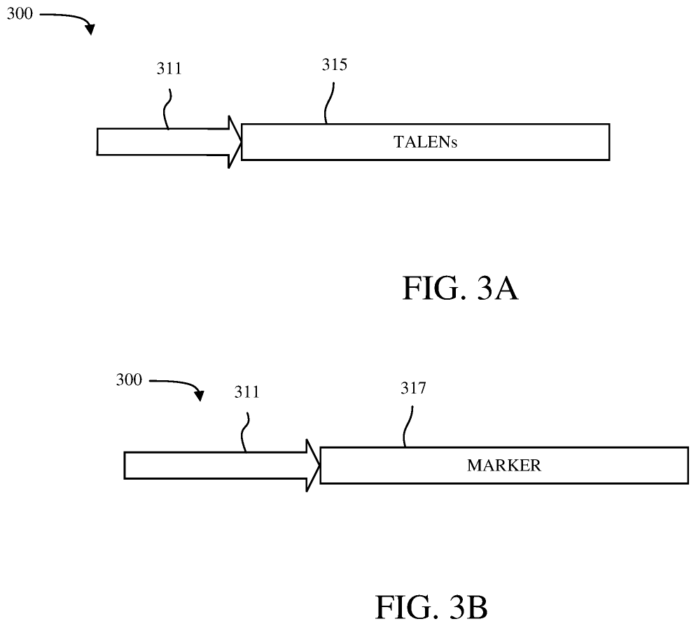

The expression construct may include a variety of nucleic acid segments, selected and arranged to facilitate long-distance transport of genome editing reagents in the phloem of the host plant. For instance, the expression construct may include a TALEN mRNA. In some examples, the expression construct may include an mRNA coding sequence and a promoter. In some examples, the expression construct includes a promoter and a marker which may enable detection of transported mRNA and/or transported protein. Example expression constructs are illustrated in FIG. 3 and discussed further herein.

Accordingly, the expression construct used to generate the chimeric plants may include a number of components, such as an mRNA coding sequence, and a promoter among other non-limiting components. Individual chimeric plants may be screened and selected for high expression of the TALEN mRNA and/or protein using end-point reverse transcriptase PCR (RT-PCR), quantitative real-time PCR (qRT-PCR) or florescent protein reporter expression (i.e., GUS, RFP or YFP) in transgenic hairy roots. For instance, in some examples, the phloem-mobile RNA included in the expression construct is GAL. In some examples, the phloem-mobile RNA may be KNOX. Similarly, in some examples, the promoter is SUT1. In some examples, the promoter may be SUC2. The promoter is not limited to the particular examples listed. A different promoter may be used, as discussed herein. As discussed further with regards to FIG. 3, the promoter may be upstream from the mRNA coding sequence. Examples are not so limited, and additional and/or different expression constructs are contemplated.

At 103, the method 100 includes screening new growth from the chimeric plant for gene edits resulting from genomic editing by the expression construct. In some examples, creating a chimeric plant includes selecting transgenic hairy roots resulting from the expression construct using a plant selective agent (i.e., spectinomycin). In some examples, sink tissues with detectable transgenic mRNA and/or protein may be screened for edits and used for further propagation to stabilize edits. In some examples, chimeric plants may be used for seed production and the seed screened for edited progeny, depending on the species.

In some examples, the method 100 may include screening the new growth from the chimeric plant with detectable transgenic mRNA and/or protein, and propagating tissue containing the detectable transgenic mRNA and/or protein.

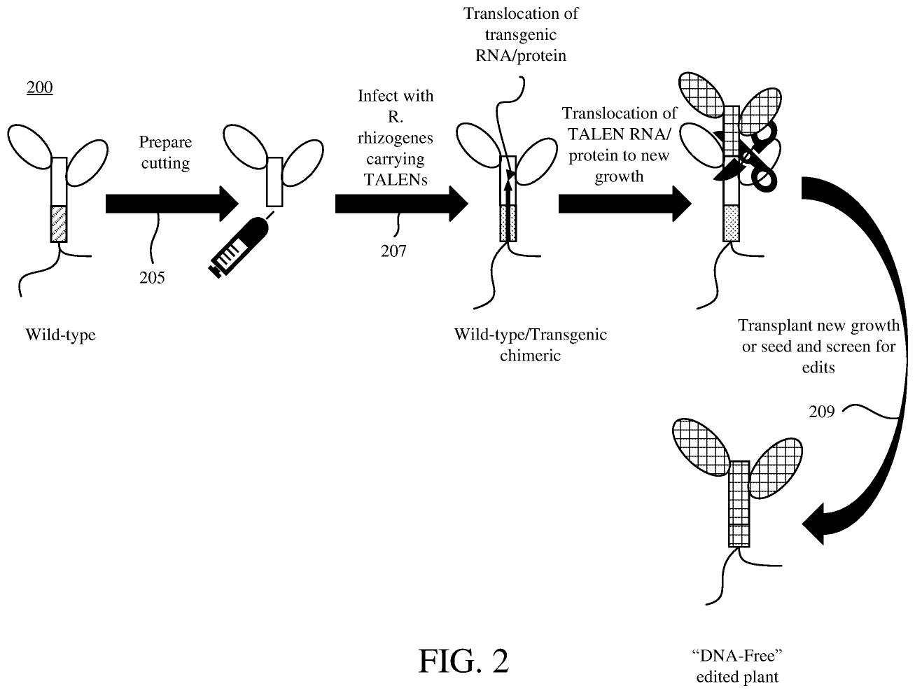

FIG. 2 illustrates an example method 200 for delivery of genome editing reagents via chimeric plants, consistent with the present disclosure. In FIG. 2, the method 200 includes, at 205, preparing a wild-type shoot or seedling excised from a host plant. Using a sterilized scissors or scalpel blade, forceps, and stereoscope, the wild-type shoot may be cut at the stem and a seedling may be cut at the hypocotyl.

At 207, the method 200 includes inoculating the wild-type shoot or seedling cutting with an R. rhizogenes solution. In accordance with the present disclosure, an mRNA of interest may be targeted for long-distant transport by positioning the mRNA's coding sequence downstream to a promoter. An example of such mRNA includes TALEN mRNA. Once such a construct is available, R. rhizogenes strains carrying the binary vector may be prepared and used to infect wild-type shoot cuttings. Non-limiting examples of R. rhizogenes strains which may be used include A4, ATCC 15834, ATCC 43056, ATCC 43057, ATCC 13333, or K599. Selection of transgenic hairy roots using a plant selective agent (i.e., spectinomycin) may allow for the formation of high expressing transgenic hairy roots (versus non-transgenic hairy roots carrying only the Ri plasmid T-DNA or wild-type roots) and increase the expression of genome editing reagents in root tissues.

As illustrated in FIG. 2, translocation of transgenic mRNA and/or protein is promoted through the wild-type/transgenic chimeric plant, from the transgenic hairy roots to the wild-type stem. Once transgenic hairy roots are established and a chimeric plant is created, transgenic mRNA and/or protein may move from transgenic hairy roots to the growing wild-type tissues, allowing editing in the wild-type tissues in the absence of transgenic DNA. As new growth develops on the chimeric plant, the transgenic mRNA and/or protein is functional in the new growth, as indicated in FIG. 2.

At 209, the method 200 includes transferring cuttings infected with the R. rhizogenes solution to a medium for growth of transgenic hairy roots. The degree of editing in the chimeric plant may be related to the abundance of transgenic mRNA and/or protein in sink tissues and may be tracked using various methods of mRNA and protein detection. For instance, chimeric plants may be assayed for accumulation of transgenic RNA and/or protein in new shoot tissue (i.e., new meristems, leaves, branches and/or inflorescences). New shoot growth may be sampled for detection of TALEN mRNA and/or protein using end-point reverse transcriptase PCR (RT-PCR) or western blot, respectively. Shoot growth positive for TALEN mRNA and/or protein may be screened for detection of edits using Illumina® amplicon sequencing of the TALEN target gene. Shoot growth positive for edits may be propagated either vegetatively or through seed to stabilize edits in individual plants, depending on the species.

Various examples of the present disclosure relate to a non-naturally occurring plant generated by the method 100 described with regards to FIG. 1 and/or the method 200 described with regards to FIG. 2. Similarly, the present disclosure relates to a non-naturally occurring seed, reproductive tissue, or vegetative tissue generated by the method 100 described with regards to FIG. 1 and/or the method 200 described with regards to FIG. 2. Various examples of the present disclosure relate to a wild-type/transgenic plant generated by the method 100 described with regards to FIG. 1 and/or the method 200 described with regards to FIG. 2. For instance, consistent with methods 100 and 200, a non-naturally occurring plant may be generated by a DNA-free genomic editing technique. The DNA-free genomic editing technique may include preparing a cutting of a host plant, generating a chimeric plant by infecting the cutting of the host plant with an R. rhizogenes solution including a TALEN mRNA coding sequence, and a promoter, screening growth from the chimeric plant resulting from genomic editing by the TALEN mRNA and/or protein, and transplanting the growth from the chimeric plant to medium for propagation or setting seed.

FIGS. 3A and 3B illustrate examples of expression constructs 300 for DNA-free delivery of genome editing reagents, consistent with the present disclosure. FIG. 3A illustrates an expression construct 300 comprising an mRNA coding sequence 315 and a promoter 311 upstream from the mRNA coding sequence 315. Non-limiting examples of mRNA coding sequences used may include TALEN mRNA coding sequence targeting a Cannabis sativa phytoene desaturase (CsPDS) gene, Cannabis sativa tetrahydrocannabinolic acid synthase (CsTHC) gene, and the Solanum tuberosum phytoene desaturase (StPDS) gene. In some examples, the expression construct 300 may include a second zip-code element (not illustrated) from a phloem-mobile RNA. For instance, one or more zip-code elements may be incorporated, upstream from the mRNA coding sequence, downstream from the mRNA coding sequence, or both upstream and downstream from the mRNA coding sequence. As may be appreciated, upstream can include a location proximal to and/or closer to the 5′ end of the promoter 311 as compared to the referenced sequence. Conversely, downstream can include a location proximal to and/or closer to the 3′ end of terminator sequence 325 as compared to the referenced sequence.

As discussed with regards to FIG. 2, the zip-code elements may be from a different phloem-mobile RNA and/or from the same phloem-mobile RNA. Moreover, each of the zip-code elements may be a UTR from the phloem-mobile RNA or full-length sequence. As non-limiting examples, the phloem-mobile RNA is GAI and/or KNOX. As non-limiting examples, the phloem-specific promoter is SUT1 and/or SUC2.

FIG. 3B illustrates an example expression construct 300 including a single mRNA sequence and a detectable marker 317 which may allow detection of the mRNA sequence and/or protein in transgenic tissue. Non-limiting examples of a detectable marker include β-glucuronidase (GUS) or a florescent protein reporter such as RFP or YFP.

Various embodiments are implemented in accordance with the underlying provisional application, U.S. Provisional Application No. 63/088,790, filed on Oct. 7, 2020, and entitled “Delivery of Genome Editing Reagents via Chimeric Plants”, to which benefit is claimed and which is fully incorporated herein by reference in entirety. Embodiments discussed in the provisional application is not intended, in any way, to be limiting to the overall technical disclosure, or to any part of the claimed invention unless specifically noted.

Publications cited herein and the subject matter for which they are cited are hereby specifically incorporated by reference in their entireties.

The skilled artisan would recognize that various terminology as used in the Specification (including claims) connote a plain meaning in the art unless otherwise indicated. The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

Based upon the above discussion and illustrations, those skilled in the art will readily recognize that various modifications and changes may be made to the various embodiments without strictly following the exemplary embodiments and applications illustrated and described herein. For example, methods as exemplified in the Figures may involve steps carried out in various orders, with one or more aspects of the embodiments herein retained, or may involve fewer or more steps. Such modifications do not depart from the true spirit and scope of various aspects of the disclosure, including aspects set forth in the claims.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

### Experimental/More Detailed Embodiments

As further illustrated below in connection with the experimental embodiments, genome editing reagents may be delivered via chimeric plants. Consistent with the above description, R. rhizogenes strains and plant source material were prepared as follows. Five to seven days prior to the experiment, the desired R. rhizogenes strain was streaked out onto media plated agar medium with antibiotics. The plates were incubated at approximately 28° C. until the day of the experiment. Six (6) days prior to the experiment, 50-100 hemp seeds were surface sterilized with approximately 10 mL concentrated sulfuric acid and washed twice with sterile water. The seeds were soaked in 30% H₂O₂ for 20 minutes and washed twice with sterile water. The seeds were allowed to imbibe in sterile water overnight (e.g., for approximately 16-24 hours) with some gentle agitation (either in a conical tube placed in a motorized invertor or a sealed petri dish on a rotary shaker). On the following day, the water was removed, and the imbibed seeds were washed one more time for approximately 5 min in 30% hydrogen peroxide before rinsing three times with sterile water. Using forceps and a stereomicroscope, the seed coats and endosperm were removed before plating the embryos onto MS (Murashige & Skoog) media plates with a maximum of five embryos per plate. The plates were sealed with parafilm and placed in the dark for 3 days. The plates were transferred to a 16/8-hour light/dark incubator (75 lumens, approximately 23° C.) for two additional days.

Infection of hemp hypocotyl tissue for hairy root production was performed as follows. Five hours prior to infection, a loopful of bacteria from the plates was suspended in approximately 1 mL of sterile water containing approximately 100 μM acetosyringone. The bacterial suspension was maintained in a dark lab drawer at room temperature. The hemp seedlings were removed from the incubator and the following steps were performed. To infect whole seedlings, the point of a scalpel was used to make a small wound in the hypocotyl of each seedling, approximately 5-10 mm above the top of the radicle. Immediately after wounding, the wound was inoculated with approximately 20 μL of bacterial suspension. After a minimum of 10 min, the seedling was transferred to a hemp hairy root co-cultivation medium. The number of seedlings per plate was limited to five inoculated seedlings. The plates were sealed with parafilm and placed in the dark overnight (approximately 22° C. to approximately 23° C.). The plates were removed from the dark chamber and transferred to a 16/8-hour light/dark incubator (75 lumens, approximately 23° C.) for one additional day for a total of two days of co-cultivation.

In the case of whole seedlings, each seedling was transferred to an individual hemp hairy root media plate containing approximately 500 mg/L of cefotaxime, and care was exercised to ensure that the previously wounded part of the hypocotyl was touching the medium. The Phytotrays™ were closed and placed in a light chamber for two weeks. Propagated new shoot growth in tissue culture or soil from rooted scions were sampled to isolate individual genome edited lines.

R. rhizogenes and the infection solution were prepared as follows. Five to seven days prior to the experiment, the desired R. rhizogenes strain was streaked out onto an LB (Luria-Bertani) media agar plate with antibiotics. The plates were incubated in an inverted position at approximately 28° C. until the day of the experiment. The day before the experiment, a single colony was inoculated into approximately 5 mL LB media with antibiotics in a 15 mL sterile culture tube and incubated with shaking at approximately 28° C. at approximately 250 rpms for approximately 16 hours. The day of the experiment, the contents of the culture were poured into approximately 50 mL LB media with antibiotics in a 50 mL sterile culture tube and incubated with shaking at approximately 28° C. at approximately 250 rpms until an O.D.600 of 0.5-0.6. Cells were collected by centrifugation at approximately 6000 rpms for approximately 10 minutes and re-suspended in Minimal Growth (MG) media. Approximately 1 mL of R. rhizogenes infection solution was dispensed into 1.5 mL tubes and approximately 100 μM of acetosyringone was added approximately 1 hour prior to infection.

Preparation and infection of soy scions for production of transgenic hairy root chimeric plant was performed as follows. Two to three weeks prior to the experiment, soy seeds were sterilized by putting the seed in a 9 L desiccator chamber with approximately 100 mL bleach in a 250 mL beaker. Approximately 3.5 mL concentrated HCl was slowly added and the desiccator chamber was kept closed for approximately 16 hours for chlorine gas sterilization. The chlorine gas was allowed to dissipate prior to seed use. Sterilized seeds were transferred to Phytatrays™ with MS media by submerging seeds and directing the seed hilum downward. The Phytatrays™ were placed under 16/8-hour light/dark (75 lumens, approximately 28° C.). Once seedlings reached the VC stage (vegetative cotyledons), cotyledons were removed from all seedlings with a sterile scalpel and aseptic technique. Seedling scions were prepared by excising the shoot, abaxial to the cotyledon node across the hypocotyl cross section and the cut end was placed into the R. rhizogenes infection solution. Seedling scions were incubated at approximately 23° C. for approximately 15 minutes. Infected cut ends were blotted on sterile filter paper and placed in Phytatrays™ with MS media by submerging the cut ends approximately 1.5 cm under the media surface. The Phytatrays™ were closed and placed under 16/8-hour light/dark (75 lumens, approximately 28° C.) at approximately 28° C. for 2 days. The infected scions were transferred to Phytatrays™ containing MS media with approximately 500 mg/L cefotaxime and approximately 150 mg/L timentin and a plant selective agent by submerging the cut ends approximately 1.5 cm under the media surface. The Phytatrays™ were closed and placed in the dark at approximately 28° C. for 2 days. New shoot growth was sampled and propagated in tissue culture or soil from rooted scions to isolate individual genome edited lines.

Preparation and infection of potato scions for production of transgenic hairy root chimeric plant was performed as follows. Approximately 3-4 weeks prior to the experiment, tissue culture potato plantlets were propagated in Magenta™ boxes with MMS (modified Murashige & Skoog) media using plantlet shoot tips and aseptic technique. The tissue culture potato plantlets were placed under 16/8-hour light/dark (75 lumens, approximately 23° C.). Once plantlets reached the 5-node stage, fully emerged leaves basal to the 4th node were removed, leaving a single node of fully emerged leaves and the apical meristem. Scion cuttings from the potato plantlets were prepared by excising the shoot, abaxial to the 4th node by making across the stem cross section and placing the cut end into the R. rhizogenes infection solution. The scion cuttings were incubated at approximately 23° C. for approximately 15 minutes. Infected cut ends were blotted on sterile filter paper and placed in Phytatrays™ with MS media by submerging cut ends approximately 1.5 cm under the media surface. The Phytatrays™ were closed and placed under 16/8-hour light/dark (75 lumens, approximately 28° C.) at approximately 28° C. for 2 days. Infected scions were transferred to Phytatrays™ containing MS media with approximately 500 mg/L cefotaxime and approximately 150 mg/L timentin and a plant selective agent by submerging cut ends approximately 1.5 cm under the media surface. The Phytatrays™ were closed and placed in the dark at approximately 28° C. for 2 days. New shoot growth was sampled and propagated in tissue culture or soil from rooted scions to isolate individual genome edited lines.

To create the MS media (Phytatrays™ or plates), the following protocol and volumes were used to make a 1 L solution of media:
1600 ml ddH2O;
10 g Sucrose;
4.43 g MS Basal Salts+Vitamins (Phytotech, M519);
The solution was brought to volume with 1000 ml ddH2O;
The pH was adjusted to 5.7 with titration of KOH;
3.58 g Gelzan™ (Phytotech, G3251);
   The media was autoclaved on the liquid cycle for 25 minutes and cooled to 55° C. and poured 100 mL per Phytatray™ or 100×25 mm plates.

To create the MMS media (Magenta boxes™), the following protocol and volumes were used to make a 1 L solution of media:
800 ml of ddH₂O;
25 g Sucrose;
4.43 g of MS Basal Salts+Vitamins (Phytotech, M519);
The solution was brought to volume with 1000 ml ddH₂O;
The pH was adjusted to 5.7 by titration of KOH
7.5 g of Agar (Phytotech, A296);
The media was autoclaved on liquid cycle for 25 minutes;
0.8 ml of Cefotaxime (250 mg/ml);
0.1 ml of 6-BAP (lmg/ml);
   The media was cooled to 55° C. and poured 100 mL per Magenta boxes™ (Sigma, V8505).

To create the (Luria-Bertani) (LB) media (culture tubes), the following protocol and volumes were used to make a 1 L solution of media:
600 ml of ddH₂O;
25 g of LB (Sigma: L3522);
The solution was brought to volume with 1000 ml of ddH₂O;
   The media was autoclaved on liquid cycle for 25 minutes.

To create the LB agar media (plates), the following protocol and volumes were used to make a 1 L solution of media:
600 ml of ddH₂O;
25 g of LB (Sigma: L3522);
15 g of Agar (Sigma: A5306);
The solution was brought to volume with 1000 ml of ddH₂O;
   The media was autoclaved on liquid cycle for 25 minutes and cooled to 55° C. and poured into 100×15 mm plates.

To create MG (minimal growth) Salts (20×), the following protocol and volumes were used:
700 ml of ddH₂O;
20 g of NH₄Cl;
6 g of MgSO₄*7H₂O;
3 g of KCl;
0.2 g of CaCl₂;
50 mg of FeSO₄*7H₂O;
   The solution was brought to volume with 1000 ml of ddH₂O;

To create MG (minimal growth) Buffer (20×), the following protocol and volumes were used to form 1 L of the media:
700 ml of ddH₂O;
60 g of K₂HPO₄;
20 g of NaH₂PO₄;
   The solution was brought to volume with 100 ml of ddH₂O.

To create liquid agar media, the following protocol and volumes were used to form 1 L of media:
700 ml of ddH₂O;
5 g of Glucose;
The solution was brought to volume with 1000 ml of ddH₂O;
50 ml of 20× MG Salts;
50 ml of 20× MG Buffer;
   The media was autoclaved on liquid cycle for 25 minutes.

To create plated agar media, the following protocol and volumes were used to form
1 L of media:
700 ml of ddH₂O;
5 g of Glucose;
The solution was brought to volume with 1000 ml of ddH₂O;
50 ml of 20× MG Salts;
50 ml of 20× MG Buffer;
15 g of Agar (Sigma: A5306);
   The media was autoclaved on liquid cycle for 25 minutes and cooled to 55° C. and poured into 100×15 mm plates.

To create hemp hairy root media (Phytatrays™), the following protocol and volumes were used:
800 ml of ddH₂O;
30 g of Sucrose (Phytotech: S9378);
4.43 g of MS Basal Salts+Vitamins (Phytotech: M519);
The solution was brought to volume with 1000 ml of ddH₂O;
The pH was adjusted to 5.8 by titration of KOH
6 g of Agarose (Phytotech: A6013);
   The media was autoclaved on liquid cycle for 25 minutes and cooled to 55° C. and poured 100 mL per Phytatray™.

To create hemp hairy root co-cultivation media (plates), the following protocol and volumes were used to create 1 L of media:
800 ml of ddH₂O;
30 g of Sucrose (Phytotech: S9378);
4.43 g of MS Basal Salts+Vitamins (Phytotech: M519);
The solution was brought to volume with 1000 ml of ddH₂O;
The pH was adjusted to 5.8 by titration of KOH;
6 g of Agarose (Phytotech: A6013);
   The media was autoclaved on liquid cycle for 25 minutes and cooled to 55° C. and poured 50 mL per 100×25 mm plate.

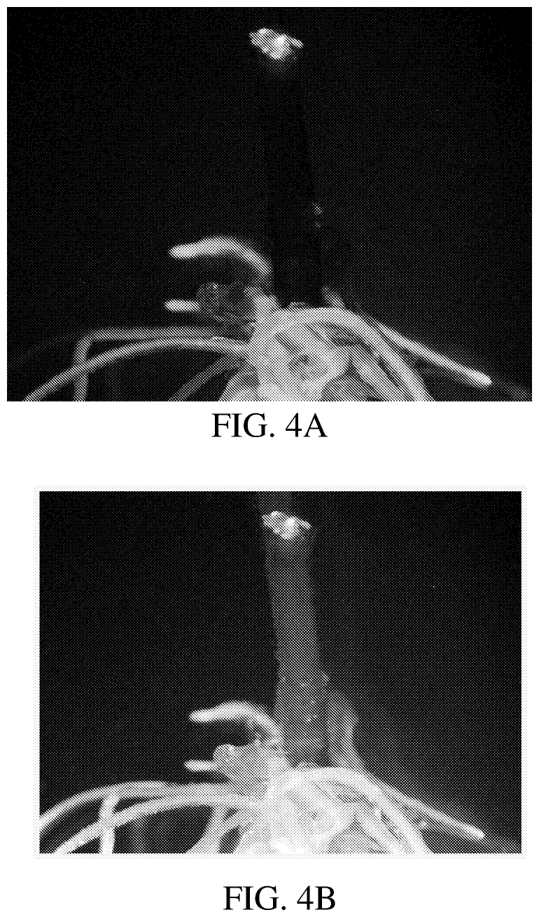

FIGS. 4A and 4B illustrate growth of transgenic hairy roots in potato, consistent with the present disclosure. Specifically, FIG. 4A illustrates fluorescent images of transgenic hairy roots in potato expressing TALEN, as discussed with regards to FIG. 3A. The transgenic tissue is illustrated as yellow fluorescent protein (YFP). FIG. 4B illustrates fluorescent and white light images of transgenic hairy roots in potato expressing TALEN. The transgenic tissue is illustrated as yellow fluorescent protein (YFP).

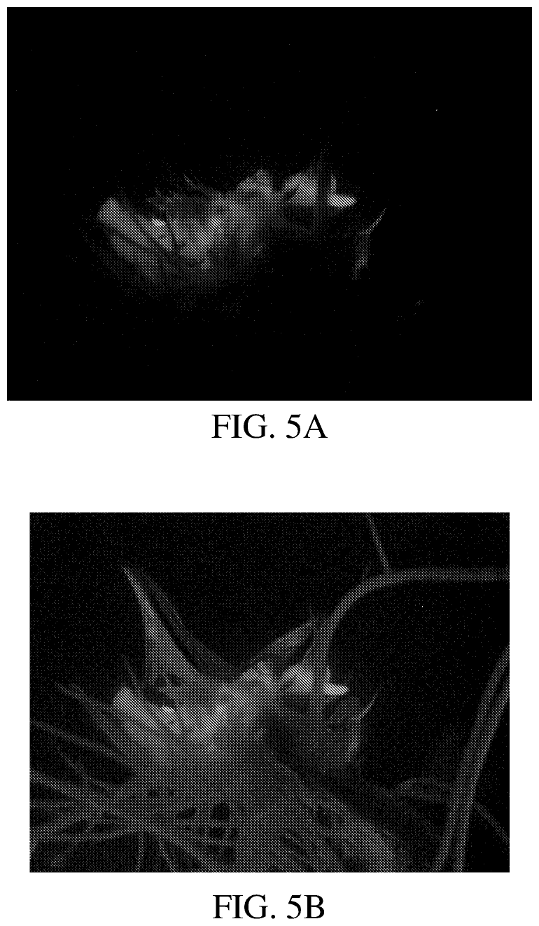

FIGS. 5A and 5B illustrate growth of transgenic hairy roots in hemp, consistent with the present disclosure. Specifically, FIG. 5A illustrates fluorescent images of transgenic hairy roots in hemp expressing TALEN, as discussed with regards to FIG. 3A. The transgenic tissue is illustrated as yellow fluorescent protein (YFP). FIG. 5B illustrates fluorescent and white light images of transgenic roots in hemp expressing TALEN. The transgenic tissue is illustrated as yellow fluorescent protein (YFP).

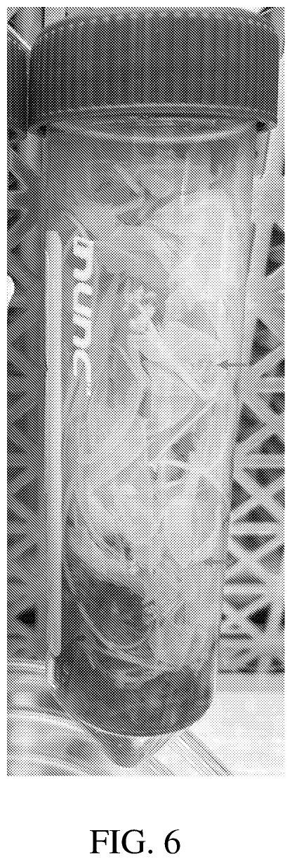

FIG. 6 illustrates chimeric hemp plant with transgenic hairy roots, consistent with the present disclosure. Specifically, FIG. 6 illustrates results of a control experiment in which hairy roots in hemp were grown without the TALEN and with a GUS marker to indicate movement of the mRNA and/or protein. As illustrated in FIG. 6, without the TALEN, the marker mRNA and/or protein was consolidated in the hairy root tissue which resulted in the blue staining in the lower portion of the plant. Few spots of staining, identified by red arrows, indicate minimal movement of the marker mRNA and/or protein to the leaves of the hemp plant.

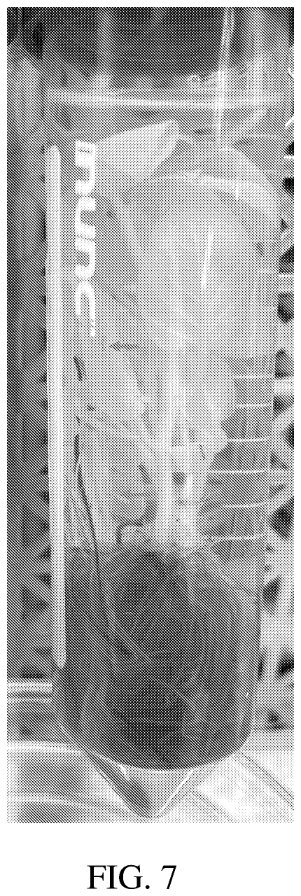

FIG. 7 illustrates chimeric potato plant with transgenic hairy roots, consistent with the present disclosure. Specifically, FIG. 7 illustrates results of a control experiment in which hairy roots in potato were grown without the TALEN and with a GUS marker to indicate movement of the mRNA and/or protein. As illustrated in FIG. 7, without the TALEN, the marker mRNA and/or protein was consolidated in the hairy root tissue which resulted in the blue staining in the lower portion of the plant. Few spots of staining, identified by red arrows, indicate minimal movement of the marker mRNA and/or protein to the leaves of the hemp plant.

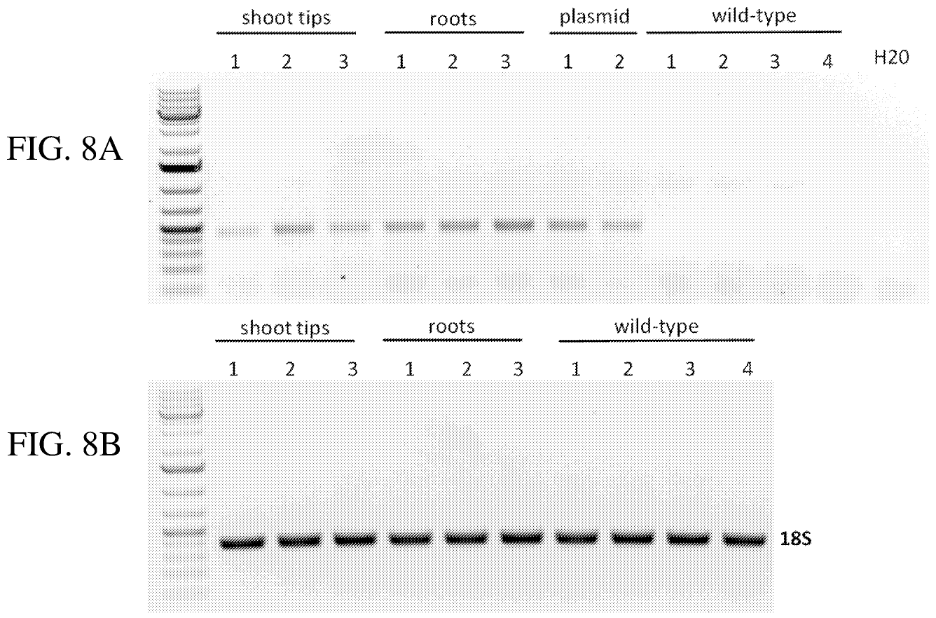

FIGS. 8A and 8B illustrate gel electrophoresis results obtained from chimeric potato plant tissue, consistent with the present disclosure. Specifically, FIG. 8A illustrates gel electrophoresis results obtained from plants that were exposed to R. rhizogenes carrying TALENs, and FIG. 8B illustrates gel electrophoresis results obtained from the samples used in 8A but detecting the control 18S gene.

As illustrated in FIG. 8A, columns were divided such that three samples of chimeric plant shoot tips were sampled (i.e., columns 1, 2, and 3 under “shoot tips”), three samples of transgenic hairy roots were sampled (i.e., columns 1, 2, and 3 under “roots”), two samples of plasmid were sampled (i.e., columns 1 and 2 under “plasmid”), four shoot tip samples of wild-type plants without R. rhizogenes carrying TALENs were sampled (i.e., columns 1, 2, 3, and 4 under “wild-type”), and a water control was used. As illustrated in FIG. 8A, all of the shoot tips, all of the roots, and the plasmid include results at 420 base pairs, corresponding with the TALEN sequence. In contrast, the wild-type plant and water results did not include any signal at 420 base pairs, indicating that the TALEN sequence was not present in the wild-type tissue or water. These results confirm that in accordance with the examples discussed herein, chimeric plants may be generated by infecting a host plant with R. rhizogenes carrying a TALEN.

FIG. 8B illustrates gel electrophoresis results from another control experiment. Columns were divided such that three samples of chimeric plant shoot tips were sampled (i.e., columns 1, 2, and 3 under “shoot tips”), three samples of transgenic hairy roots were sampled (i.e., columns 1, 2, and 3 under “roots”), four shoot tip samples of wild-type plants were sampled (i.e., columns 1, 2, 3, and 4 under “wild-type”). The plant tissue samples used in FIG. 8B are the same that were used in 8A, and all plant tissue sampled carry an 18S gene, which is found across plants. The 18S gene acted as a positive control for the RNA samples used in the RT-PCR. The results were that each sample displayed results at 493 base pairs and had comparable RNA quality. The combination of results illustrated in FIGS. 8A and 8B demonstrate that R. rhizogenes carrying an expression construct including a TALEN allows for mRNA and/or protein to travel to shoot tips from transgenic hairy roots of chimeric plants as discussed herein.

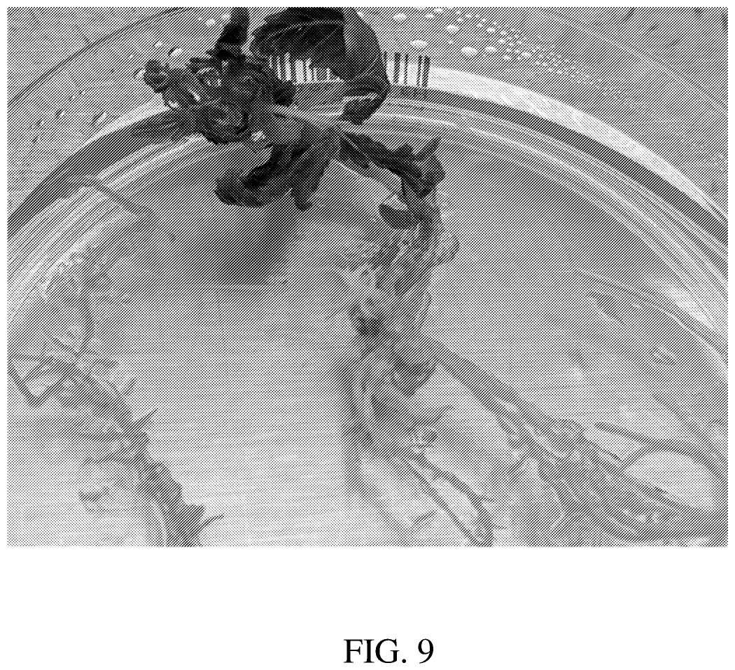

FIG. 9 illustrates development of transgenic hairy roots, consistent with the present disclosure. Particularly, FIG. 9 illustrates an example of a whole hemp seedling starting to form hairy roots at the wound site, indicated by the red arrow.

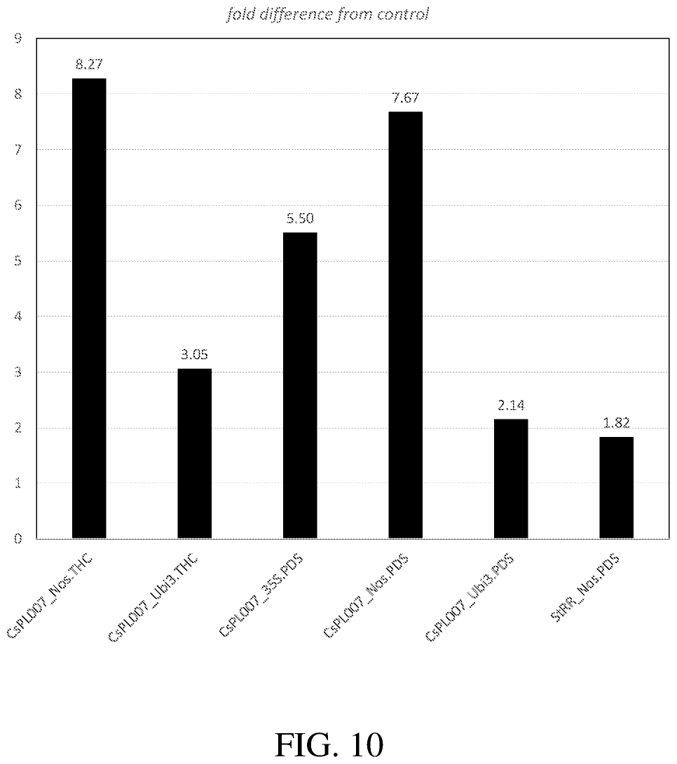

FIG. 10 illustrates data obtained from shoot tips of chimeric potato plants, consistent with the present disclosure. Specifically, FIG. 10 illustrates the fold increase of genomic editing in shoot tips of chimeric plants. The first column (from the left) illustrates that the CsPL007_Nos.THC chimeric hemp demonstrated an 8.27 fold increase in genomic editing of the THC gene in shoot tips over wild-type hemp, and the second column illustrates that the CsPL007_Ubi3.THC chimeric hemp demonstrated a 3.05 fold increase in genomic editing of the THC gene in shoot tips over wild-type hemp. The third column illustrates that the CsPL007_35S.PDS chimeric hemp demonstrated a 5.50 fold increase in genomic editing of the PDS gene in shoot tips over wild-type hemp, the fourth column illustrates that the CsPL007_Nos.PDS chimeric hemp demonstrated a 7.67 fold increase in genomic editing of the PDS gene in shoot tips over wild-type hemp, and the fifth column illustrates that the CsPL007_Ubi3.PDS chimeric hemp demonstrated a 2.14 fold increase in genomic editing of the PDS gene in shoot tips over wild-type hemp. The sixth column illustrates that the StRR_Nos.PDS s chimeric potato demonstrated a 1.82 fold increase in genomic editing of the PDS gene in shoot tips over wild-type potato.

1. A method of delivery of genome editing reagents, comprising:
generating a chimeric plant by infecting a cutting of a host plant with Rhizobium rhizogenes carrying an expression construct; and
screening new growth from the chimeric plant for gene edits resulting from the expression construct.

2. The method of claim 1, wherein the chimeric plant includes a wild type/transgenic chimeric plant.

3. The method of claim 1, wherein the expression construct includes a transcription activator like effector nuclease (TALEN) mRNA.

4. The method of claim 1, wherein the expression construct includes an mRNA coding sequence and a promoter.

5. The method of claim 4, wherein the promoter is 35S.

6. The method of claim 4, wherein the promoter is nopaline synthase (Nos).

7. The method of claim 1, including:
screening the new growth from the chimeric plant with detectable transgenic mRNA or protein from creation of the chimeric plant or genomic edits from the genomic editing; and
propagating tissue or seed from the detectable transgenic mRNA or protein or genomic edits.

8. A method of generating chimeric plants expressing genome editing reagents, comprising:
preparing a wild-type shoot or seedling cutting from a host plant;
inoculating the wild-type shoot or seedling cutting with a Rhizobium rhizogenes solution; and
transferring the wild-type shoot or seeding infected with the R. rhizogenes solution to a medium for growth of transgenic hairy roots.

9. The method of claim 8, including screening and selecting individual chimeric plants for expression of transcription activator like effector nuclease (TALEN) mRNA.

10. The method of claim 8, including detecting accumulation of transgenic mRNA and/or protein in shoot tissue using end-point reverse transcriptase PCR (RT-PCR) or western blot.

11. A non-naturally occurring plant generated by the method of claim 8.

12. A non-naturally occurring seed, reproductive tissue, or vegetative tissue generated by the method of claim 8.

13. A non-naturally occurring plant, generated by a genomic editing technique, wherein the genomic editing technique includes:
preparing a cutting of a host plant;
generating a chimeric plant by infecting the cutting of the host plant with an Rhizobium rhizogenes solution including:
a transcription activator like effector nuclease (TALEN) messenger ribonucleic acid (mRNA) coding sequence; and
a promoter;
screening growth from the chimeric plant resulting from genomic editing for genomic edits of a target gene or by detection of the TALEN mRNA and/or protein; and
propagating the growth or seed from the chimeric plant with accumulation of the TALEN mRNA and/or protein for isolation of edited events.

14. The non-naturally occurring plant of claim 13, wherein the TALEN mRNA targets a Cannabis sativa tetrahydrocannabinolic acid synthase (CsTHC) gene.

15. The non-naturally occurring plant of claim 13, wherein the TALEN mRNA targets a Solanum tuberosum phytoene desaturase (StPDS) gene.
